(19) United States
(12) Patent Application Publication
(10) Pub. No.: US 2008/0119481 A1
(43) Pub. Date: May 22, 2008

(54) Crystalline forms of 6-methoxy-8-[4-(1-(5-fluoro)-quinolin-8-yl-piperidin-4-yl)-piperazin-1-yl]-quinoline

(75) Inventors: James J. Bicksler, Titusville, NJ (US); Jessica K. Liang, Suffem, NY (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 11/811,379

(22) Filed: Jun. 8, 2007

Related U.S. Application Data
(60) Provisional application No. 60/812,167, filed on Jun. 9, 2006.

(51) Int. Cl. A61K 31/497; C07D 401/14; A61P 25/00

(52) U.S. Cl. 514/253.06; 544/363

(57) ABSTRACT

The present invention relates to crystalline forms of the 5-HT_{1A} binding agent 6-methoxy-8-[4-(1-(5-fluoro)-quinolin-8-yl-piperidin-4-yl)-piperazin-1-yl]-quinoline, as well as pharmaceutical compositions thereof, and methods of use thereof.

### CROSS REFERENCE TO RELATED APPLICATIONS

This application claims the benefit of U.S. Provisional Patent App. Ser. No. 60/812,167, filed Jun. 9, 2006, the disclosure of which is incorporated herein by reference in its entirety.

### FIELD OF THE INVENTION

The present invention relates to crystalline forms of the 5-HT_{1A} binding agent 6-methoxy-8-[4-(1-(5-fluoro)-quinolin-8-yl-piperidin-4-yl)-piperazin-1-yl]-quinoline, as well as pharmaceutical compositions thereof, and methods of use thereof.

### BACKGROUND OF THE INVENTION

N-Aryl-piperazine derivatives are known to bind to 5-HT_{1A} receptors and are useful as pharmaceutical agents for the treatment of various central nervous system (CNS) disorders such as cognitive disorders, anxiety disorders, and depression. See, e.g., Childers, et al., J. Med. Chem., 2005, 48, 3467; and U.S. Pat. Nos. 6,465,482; 6,127,357; 6,469,007; and 6,586,436, as well as WO 97/03982. Among these, certain N-aryl-piperazine-piperidine compounds, including 6-methoxy-8-[4-(1-(5-fluoro)-quinolin-8-yl-piperidin-4-yl)-piperazin-1-yl]-quinoline (see Formula I), which is described in WO 2006/135839 have been found to modulate activity of the 5-HT_{1A} receptor and are useful, for example, for enhancing cognition, treating anxiety, and treating depression, among other CNS disorders.

Drug compounds are typically combined with other pharmaceutically acceptable ingredients to form compositions suitable for a desired mode of administration. Solid formulations often require that the drug compound have workable solid state characteristics such as stability to heat and humidity, ease of handling, and other characteristics that facilitate preparation of solid dosage forms. Accordingly, there is an ongoing need for stabler forms of existing drug molecules as well as intermediates and methods for preparing such forms. The crystalline forms of 6-methoxy-8-[4-(1-(5-fluoro)-quinolin-8-yl-piperidin-4-yl)-piperazin-1-yl]-quinoline described herein are directed toward this end.

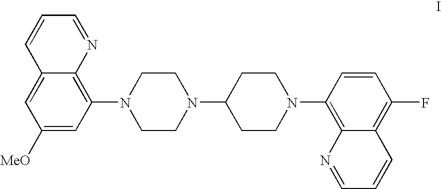

### SUMMARY OF THE INVENTION

The present invention provides crystalline forms of 6-methoxy-8-[4-(1-(5-fluoro)-quinolin-8-yl-piperidin-4-yl)-piperazin-1-yl]-quinoline.

The present invention further provides a crystalline form of 6-methoxy-8-[4-(1-(5-fluoro)-quinolin-8-yl-piperidin-4-yl)-piperazin-1-yl]-quinoline designated as Form A.

The present invention further provides a crystalline form of 6-methoxy-8-[4-(1-(5-fluoro)-quinolin-8-yl-piperidin-4-yl)-piperazin-1-yl]-quinoline designated as Form B.

The present invention further provides a crystalline form of 6-methoxy-8-[4-(1-(5-fluoro)-quinolin-8-yl-piperidin-4-yl)-piperazin-1-yl]-quinoline designated as Form C.

The present invention further provides a crystalline form of 6-methoxy-8-[4-(1-(5-fluoro)-quinolin-8-yl-piperidin-4-yl)-piperazin-1-yl]-quinoline designated as Form D.

The present invention further provides a crystalline form of 6-methoxy-8-[4-(1-(5-fluoro)-quinolin-8-yl-piperidin-4-yl)-piperazin-1-yl]-quinoline designated as Form E.

The present invention further provides compositions comprising the crystalline forms described herein.

The present invention further provides processes for the preparation of the crystalline forms described herein. The present invention further provides crystalline forms prepared by the processes of preparation described herein.

The present invention further provides methods of treating one or more of the diseases recited herein.

### BRIEF DESCRIPTION OF THE DRAWINGS

FIG. 1 depicts an XRPD pattern consistent with Form A.

FIG. 2 depicts an XRPD pattern consistent with Form B.

FIG. 3 depicts an XRPD pattern consistent with Form C.

FIG. 4 depicts an XRPD pattern consistent with Form D.

FIG. 5 depicts an XRPD pattern consistent with From E.

FIG. 6 depicts DSC traces consistent with Forms A, B, C, D, and E.

### DETAILED DESCRIPTION

### Crystalline Forms

The present invention provides crystalline forms of the compound 6-methoxy-8-[4-(1-(5-fluoro)-quinolin-8-yl-piperidin-4-yl)-piperazin-1-yl]-quinoline (see Formula I above) which can modulate the 5-HT_{1A} receptor and is useful in the treatment of CNS disorders.

As used herein, “crystalline form” is meant to refer to a certain lattice configuration of a crystalline substance. Different crystalline forms of the same substance (e.g., 6-methoxy-8-[4-(1-(5-fluoro)-quinolin-8-yl-piperidin-4-yl)-piperazin-1-yl]-quinoline) typically have different crystalline lattices (e.g., unit cells), typically have different physical properties attributed to their different crystalline lattices, and in some instances, have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the crystalline form as well as help determine stability and solvent/water content.

Different crystalline forms of a particular substance can include both anhydrous forms of that substance and solvated/hydrated forms of that substance, where each of the anhydrous forms and solvated/hydrated forms are distinguished from each other by different XRPD patterns (or different unit cells in the case of single crystal X-ray crystallography), thereby signifying different crystalline lattices. In some instances, a single crystalline form (e.g., identified by a unique XRPD pattern) can have variable water or solvent content, where the lattice remains substantially unchanged (as does the XRPD pattern) despite the compositional variation with respect to water and/or solvent.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the machine or the settings (for example, whether a Ni filter is used or not). As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.20 (2-theta), and the term “substantially” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures is understood to accommodate such variation.

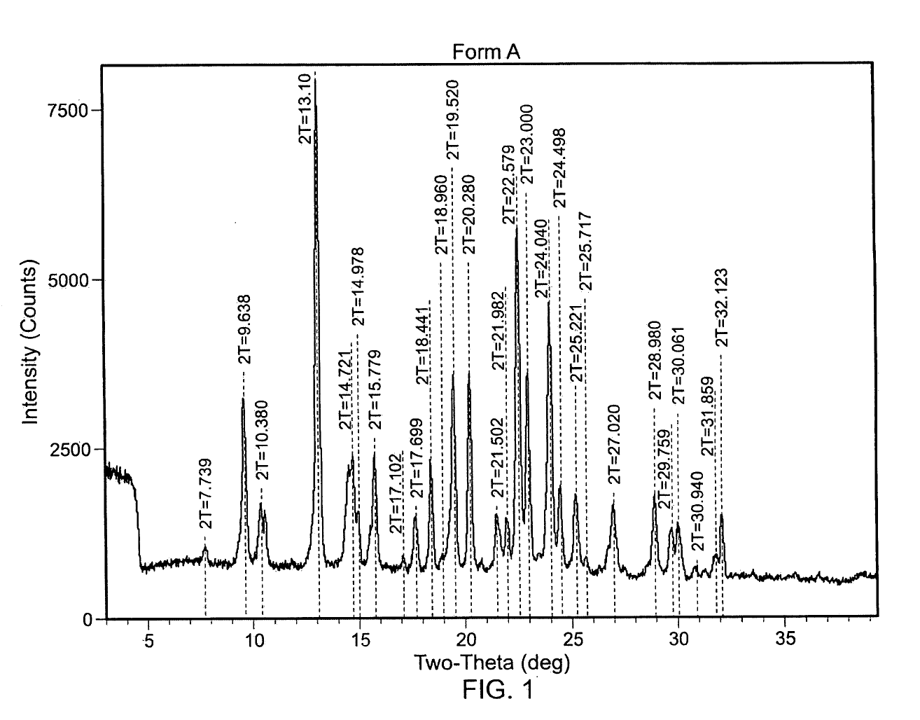

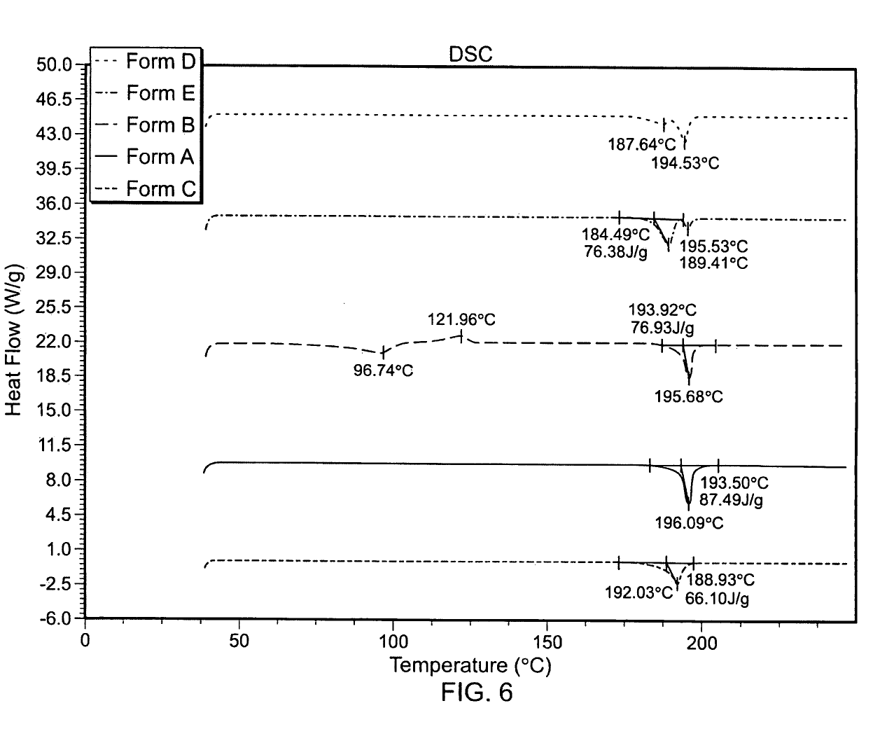

The present invention provides five crystalline forms of 6-methoxy-8-[4-(1-(5-fluoro)-quinolin-8-yl-piperidin-4-yl)-piperazin-1-yl]-quinoline free base. A first crystalline form, designated Form A, is believed to be substantially anhydrous and unsolvated with a melting point of about 196° C. Form A is characterized according to the XRPD pattern of FIG. 1 (see Table 1 for peak data) as well as the DSC data provided in FIG. 6. The DSC trace showed a single endotherm centered at about 196° C. which is attributed to a melt event.

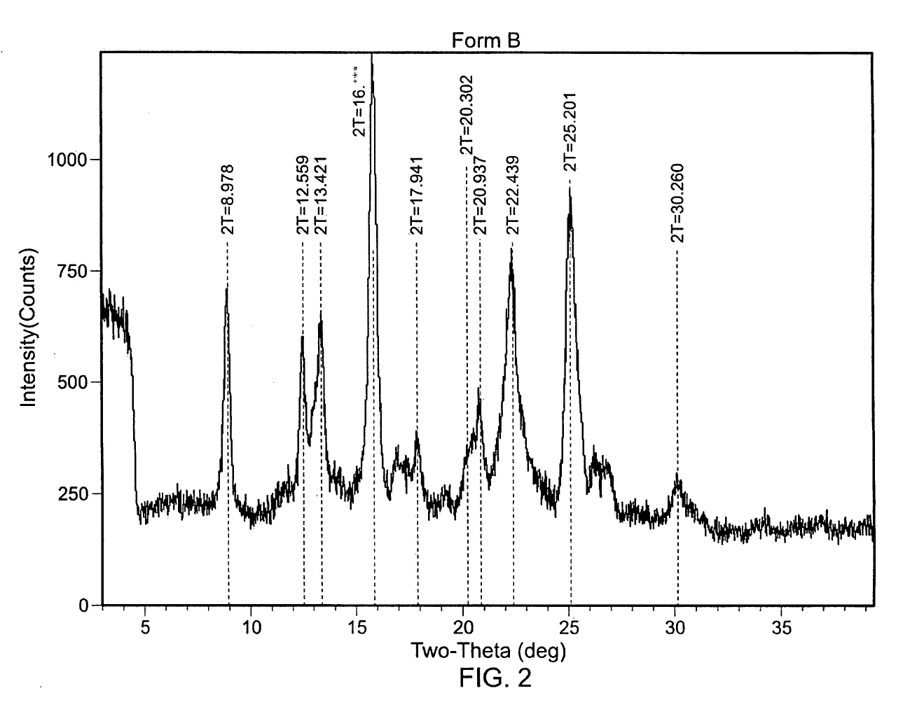

A second crystalline form, designated Form B, is believed to be a hydrate. Form B is characterized according to the XRPD pattern of FIG. 2 (see Table 1 for peak data) as well as the DSC data provided in FIG. 6. The DSC trace showed a broad endotherm at about 97° C.; an exotherm at about 122° C.; and a sharp endotherm at about 196° C.

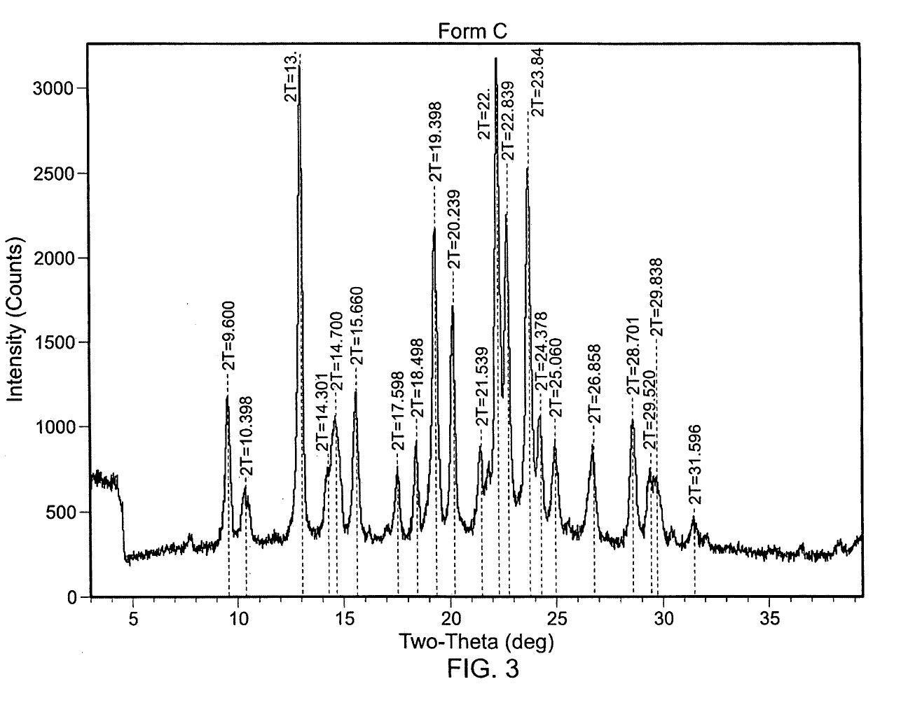

A third crystalline form, designated Form C, is believed to be substantially anhydrous and unsolvated. Form C is characterized according to the XRPD pattern of FIG. 3 (see Table 1 for peak data) as well as the DSC data provided in FIG. 6. The DSC trace showed a sharp endotherm centered at about 192° C.

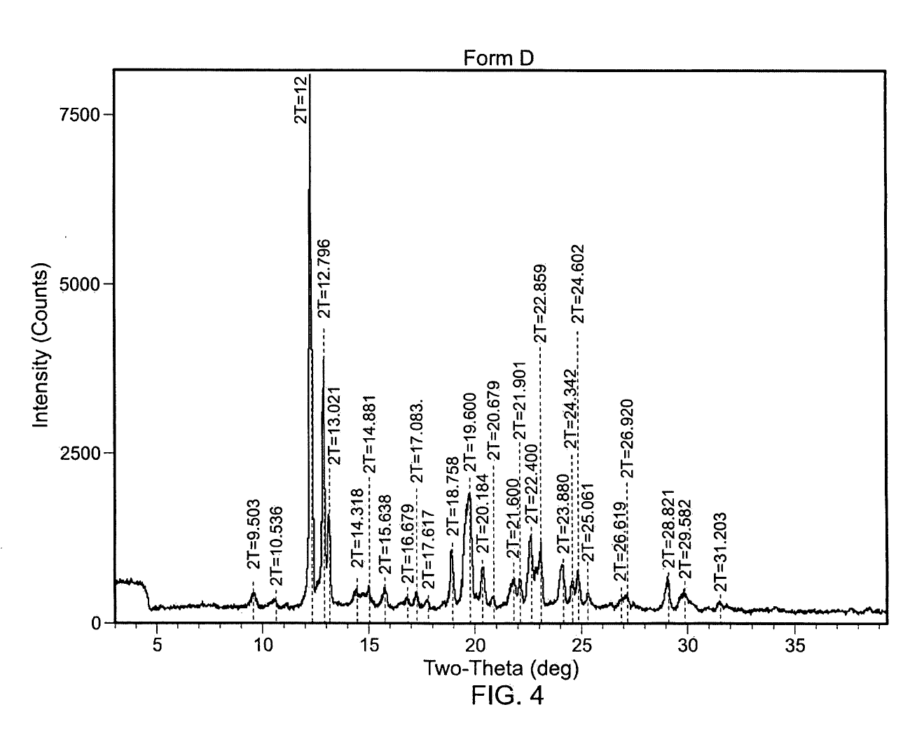

A fourth crystalline form, designated Form D, is believed to be a solvate. Form D is characterized according to the XRPD pattern of FIG. 4 (see Table 1 for peak data) as well as the DSC data provided in FIG. 6. The DSC trace showed a first broad endotherm centered at about 188° C. which is believed to correspond to a desolvation event and another broad endotherm at about 192° C.

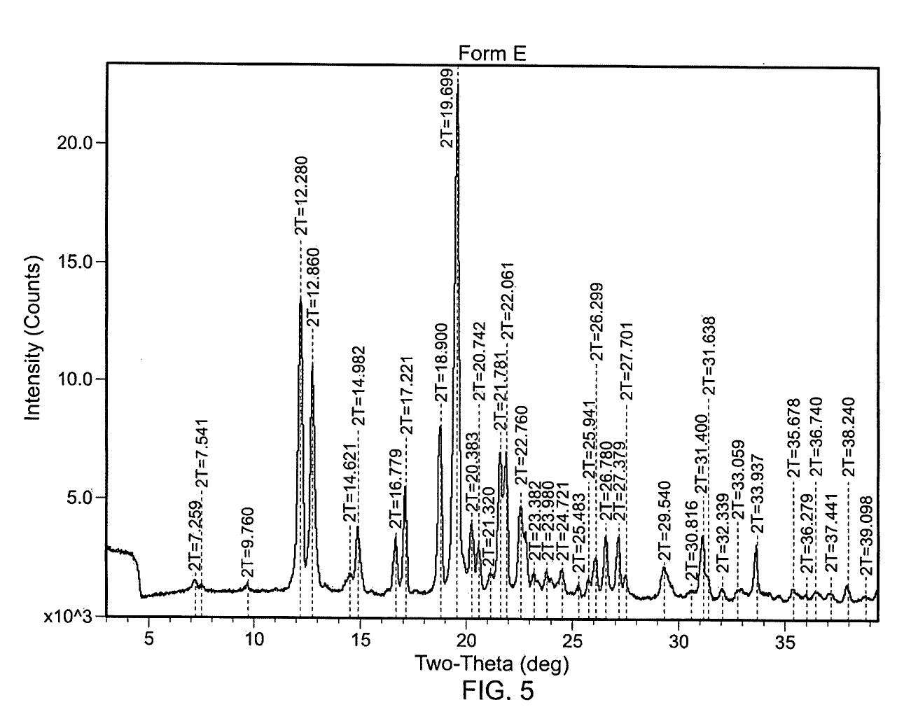

A fifth crystalline form, designated Form E, is believed to be a solvate. Form E is characterized according to the XRPD pattern of FIG. 5 (see Table 1 for peak data) as well as the DSC data provided in FIG. 6. The DSC trace showed a broad shoulder at 185° C. and broad endotherms at 189° C. and 196° C.

XRPD peak and intensity data acquired for each of the five crystalline forms are provided below in Table 1. Instrument and collection parameters are provided below in the Examples. Intensities are provided as relative intensities such that +++ represents an intensity that is equal to or greater than 50% of the maximum intensity; ++ represents an intensity that is equal to or greater than 25% of the maximum intensity but less than 50% of the maximum intensity; and +represents an intensity that is less than 25% of the maximum intensity.

TABLE 1

| Form A | | Form B | | Form C | | Form D | | Form E | |
|---|---|---|---|---|---|---|---|---|---|
| 2θ | Inten. | 2θ | Inten. | 2θ | Inten. | 2θ | Inten. | 2θ | Inten. |
| 7.7 | + | 9.0 | +++ | 9.6 | ++ | 9.5 | + | 7.3 | + |
| 9.6 | ++ | 12.6 | ++ | 10.4 | + | 10.5 | + | 7.5 | + |
| 10.4 | + | 13.4 | ++ | 13.1 | +++ | 12.2 | +++ | 9.8 | + |
| 13.1 | +++ | 15.9 | +++ | 14.3 | + | 12.8 | ++ | 12.3 | +++ |
| 14.7 | + | 17.9 | + | 14.7 | + | 13.0 | + | 12.9 | ++ |
| 15.0 | + | 20.3 | + | 15.7 | ++ | 14.3 | + | 14.6 | + |
| 15.8 | + | 20.9 | + | 17.6 | + | 14.9 | + | 15.0 | + |
| 17.1 | + | 22.4 | +++ | 18.5 | + | 15.6 | + | 16.8 | + |
| 17.7 | + | 25.2 | +++ | 19.4 | +++ | 16.7 | + | 17.2 | + |
| 18.4 | + | 30.3 | + | 20.2 | ++ | 17.1 | + | 18.9 | ++ |
| 19.0 | + | | | 21.5 | + | 17.6 | + | 19.7 | +++ |
| 19.5 | ++ | | | 22.4 | +++ | 18.8 | + | 20.4 | + |
| 20.3 | ++ | | | 22.8 | +++ | 19.6 | + | 20.7 | + |
| 21.5 | + | | | 23.8 | +++ | 20.2 | + | 21.3 | + |
| 22.0 | + | | | 24.4 | + | 20.7 | + | 21.8 | ++ |
| 22.6 | +++ | | | 25.1 | + | 21.6 | + | 22.1 | ++ |
| 23.0 | ++ | | | 26.9 | + | 21.9 | + | 22.8 | + |
| 24.0 | +++ | | | 28.7 | + | 22.4 | + | 23.4 | + |
| 24.5 | + | | | 29.5 | + | 22.9 | + | 24.0 | + |
| 25.2 | + | | | 29.8 | + | 23.9 | + | 24.7 | + |
| 25.7 | + | | | 31.6 | + | 24.3 | + | 25.5 | + |
| 27.2 | + | | | | | 24.6 | + | 25.9 | + |
| 29.0 | + | | | | | 25.1 | + | 26.3 | + |
| 29.8 | + | | | | | 26.6 | + | 26.8 | + |
| 30.1 | + | | | | | 26.9 | + | 27.4 | + |
| 30.9 | + | | | | | 28.8 | + | 27.7 | + |
| 31.9 | + | | | | | 29.6 | + | 29.5 | + |
| 32.1 | + | | | | | 31.2 | + | 30.8 | + |
| | | | | | | | | 31.4 | + |
| | | | | | | | | 31.6 | + |
| | | | | | | | | 32.3 | + |
| | | | | | | | | 33.1 | + |
| | | | | | | | | 33.9 | + |
| | | | | | | | | 35.7 | + |
| | | | | | | | | 36.3 | + |
| | | | | | | | | 36.7 | + |
| | | | | | | | | 37.4 | + |
| | | | | | | | | 38.2 | + |
| | | | | | | | | 39.1 | + |

Advantages of each of the crystalline forms is readily apparent. For example, the high melting point of Form A implies superior stability thereby imparting a relatively long shelf life to solid pharmaceutical formulations made with this crystalline form. Form C having a lower melting point would be expected to have better solubility and therefore show better bioavailability. Forms B as a hydrate would have the advantage of being able to be prepared under conditions that were not rigorously water-free, and allow the use of less hazardous aqueous solvents during preparation. Forms D and E can be useful intermediates in the preparation of Form A or additional anhydrous, unsolvated forms.

In some embodiments, the present invention provides a crystalline Form A of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 9.6 and about 13.1, and having a DSC thermogram which is characterized by an endothermic peak at about 196° C. In some embodiments, the pattern further comprises a characteristic peak, in terms of 2θ (°), at about 22.6. In further embodiment, the pattern further comprises a characteristic peak, in terms of 2θ (°), at about 24.0.

In some embodiments, the present invention provides crystalline form (Form A) of 6-methoxy-8-[4-(1-(5-fluoro)-quinolin-8-yl-piperidin-4-yl)-piperazin-1-yl]-quinoline having an X-ray powder diffraction pattern comprising at least three characteristic peaks, in terms of 2θ (°), selected from about 9.6, about 13.1, about 22.6, about 24.0, about 19.5, and about 20.3, and having a DSC thermogram which is characterized by an endothermic peak at about 196° C.

In some embodiments, the present invention provides a crystalline form (Form A) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 1.

In some embodiments, the present invention provides a crystalline form (Form A) of 6-methoxy-8-[4-(1-(5-fluoro)-quinolin-8-yl-piperidin-4-yl)-piperazin-1-yl]-quinoline having a DSC thermogram which is characterized by an endothermic peak at about 196° C.

In some embodiments, the present invention provides a crystalline form of claim 6 having a DSC thermogram substantially as shown in FIG. 6.

In some embodiments, the present invention provides a crystalline form (Form B) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 9.0 and about 15.9. In some embodiments, the pattern further comprises a characteristic peak, in terms of 2θ (°), at about 22.4. In further embodiments, the pattern further comprises a characteristic peak, in terms of 2θ (°), at about 25.2.

In some embodiments, the present invention provides a crystalline form (Form B) of 6-methoxy-8-[4-(1-(5-fluoro)-quinolin-8-yl-piperidin-4-yl)-piperazin-1-yl]-quinoline having an X-ray powder diffraction pattern comprising a characteristic peak, in terms of 2θ (°), of about 9.0 and at least three characteristic peaks, in terms of 20, selected from about 12.6, about 13.4, about 15.9, about 22.4, and about 25.2.

In some embodiments, the present invention provides a crystalline form (Form B) of 6-methoxy-8-[4-(1-(5-fluoro)-quinolin-8-yl-piperidin-4-yl)-piperazin-1-yl]-quino line having an X-ray powder diffraction pattern substantially as shown in FIG. 2.

In some embodiments, the present invention provides a crystalline form (Form B) having a DSC thermogram substantially as shown in FIG. 6.

In some embodiments, the present invention provides a crystalline form (Form C) of 6-methoxy-8-[4-(1-(5-fluoro)-quinolin-8-yl-piperidin-4-yl)-piperazin-1-yl]-quino line having an X-ray powder diffraction pattern comprising characteristic peaks, in terms of 2θ (°), at about 9.6 and about 13.1, and having a DSC thermogram which is characterized by an endothermic peak at about 192° C. In some embodiments, the pattern further comprises a characteristic peak, in terms of 2θ (°), at about 22.4. In some embodiments, the pattern further comprising a characteristic peak, in terms of 2θ (°), at about 23.8.

In some embodiments, the present invention provides a crystalline form (Form C) of 6-methoxy-8-[4-(1-(5-fluoro)-quinolin-8-yl-piperidin-4-yl)-piperazin-1-yl]-quinoline having an X-ray powder diffraction pattern comprising at least three characteristic peaks, in terms of 2θ (°), selected from about 9.6, about 13.1, about 15.7, about 19.4, about 22.4, about 22.8, about 23.8, and having a DSC thermogram which is characterized by an endothermic peak at about 192° C.

In some embodiments, the present invention provides a crystalline form (Form C) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 3.

In some embodiments, the present invention provides a crystalline form (Form C) of 6-methoxy-8-[4-(1-(5-fluoro)-quinolin-8-yl-piperidin-4-yl)-piperazin-1-yl]-quinoline having a DSC thermogram which is characterized by an endothermic peak at about 192° C.

In some embodiments, the present invention provides a crystalline form (Form C) having a DSC thermogram substantially as shown in FIG. 6.

In some embodiments, the present invention provides a crystalline form (Form D) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 12.2 and about 12.8. In some embodiments, the pattern further comprises a characteristic peak, in terms of 2θ (°), at about 13.0. In some embodiments, the pattern further comprises a characteristic peak, in terms of 2θ (°), at about 19.6.

In some embodiments, the present invention provides a crystalline form (Form D) of 6-methoxy-8-[4-(1-(5-fluoro)-quinolin-8-yl-piperidin-4-yl)-piperazin-1-yl]-quinoline having an X-ray powder diffraction pattern comprising a characteristic peak, in terms of 2θ (°), of about 12.2 and at least three characteristic peaks, in terms of 20, selected from about 12.8, about 13.0, about 18.8, about 19.6, about 22.4.

In some embodiments, the present invention provides a crystalline form (Form D) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 4.

In some embodiments, the present invention provides a crystalline form (Form D) of 6-methoxy-8-[4-(1-(5-fluoro)-quinolin-8-yl-piperidin-4-yl)-piperazin-1-yl]-quinoline having a DSC thermogram substantially as shown in FIG. 6.

In some embodiments, the present invention provides a crystalline form (Form E) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 12.3 and about 19.7. In some embodiments, the pattern further comprises a characteristic peak, in terms of 2θ (°), at about 18.9. In some embodiments, the pattern further comprising a characteristic peak, in terms of 2θ (°), at about 21.8.

In some embodiments, the present invention provides a crystalline form (Form E) of 6-methoxy-8-[4-(1-(5-fluoro)-quinolin-8-yl-piperidin-4-yl)-piperazin-1-yl]-quino line having an X-ray powder diffraction pattern comprising a characteristic peak, in terms of 2θ (°), of about 12.3 and at least three characteristic peaks, in terms of 20, selected from about 12.9, about 18.9, about 19.7, about 21.8, about 22.1.

In some embodiments, the present invention provides a crystalline form (Form E) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 5.

In some embodiments, the present invention provides a crystalline form (Form E) of 6-methoxy-8-[4-(1-(5-fluoro)-quinolin-8-yl-piperidin-4-yl)-piperazin-1-yl]-quinoline having a DSC thermogram substantially as shown in FIG. 6.

### Preparation of Crystalline Forms

The crystalline forms of the invention can be prepared by routine methods such as by precipitating the crystalline form from a solution containing 6-methoxy-8-[4-(1-(5-fluoro)-quinolin-8-yl-piperidin-4-yl)-piperazin-1-yl]-quinoline or slurrying solid 6-methoxy-8-[4-(1-(5-fluoro)-quinolin-8-yl-piperidin-4-yl)-piperazin-1-yl]-quinoline in an organic solvent, water, or mixture thereof. Precipitation can be carried out by any suitable means such as by cooling, reducing solvent volume (e.g., evaporation), or addition of antisolvent (e.g., vapor diffusion, layer diffusion, direct mixing, etc.). Solids can be collected by filtration, dried, and analyzed according to routine methods.

Crystalline Form A can be prepared by precipitation from a solution of 6-methoxy-8-[4-(1-(5-fluoro)-quinolin-8-yl-piperidin-4-yl)-piperazin-1-yl]-quinoline in various organic solvents. Suitable organic solvents include, for example, aliphatics (e.g., pentane, hexanes, heptane, etc.), halogenated aliphatics (e.g., dichloromethane), aromatics (e.g., benzene, toluene, pyridine, chlorobenzene, etc.), ketones (e.g., acetone, 2-butanone, methyl ethyl ketone etc.), ethers (diethyl ether, tetrahydrofuran, etc.), alcohols (e.g., methanol, ethanol, butanol, 2-propanol, etc.), acetates (e.g., methyl acetate), and the like and mixtures thereof. Precipitation can be induced by any of many routine methods including lowering the temperature of the solution, concentrating the solution by evaporation (e.g., under air, under gas flow, or under vacuum), seeding, addition of antisolvent, or combination of any of these techniques. In some embodiments, precipitation is induced by evaporation.

Crystalline. Form B can be prepared by precipitation from a solution of 6-methoxy-8-[4-(1-(5-fluoro)-quinolin-8-yl-piperidin-4-yl)-piperazin-1-yl]-quinoline in DMSO or similar solvent. Precipitation can be induced by addition of water which acts as an antisolvent.

Crystalline Form C can be prepared by precipitation from a solution of 6-methoxy-8-[4-(1-(5-fluoro)-quinolin-8-yl-piperidin-4-yl)-piperazin-1-yl]-quinoline in a mixture of ketone (such as methyl ethyl ketone) and dioxane. Precipitation can be induced by evaporation of solvent.

Crystalline Form D can be prepared by precipitation from a solution of 6-methoxy-8-[4-(1-(5-fluoro)-quinolin-8-yl-piperidin-4-yl)-piperazin-1-yl]-quinoline in an acetate such as ethyl acetate. Precipitation can be induced, for example, by evaporation.

Crystalline Form E can be prepared by slurrying Form A in an organic solvent such as tetrahydrofuran at elevated temperature (e.g., about 40 to about 60° C. such as about 50° C.).

The present invention further provides crystalline Forms A, B, C, D and E prepared by any of the methods described herein.

### Compositions

The present invention further provides compositions containing crystalline form of the invention and one or more other ingredients. In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of a crystalline form of the invention or mixture thereof.

In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of 6-methoxy-8-[4-(1-(5-fluoro)-quinolin-8-yl-piperidin-4-yl)-piperazin-1-yl]-quinoline as Form A, Form B, Form C, Form D, or Form E, or mixture thereof.

In some embodiments, the composition is a pharmaceutical composition which contains at least one crystalline form of the invention and at least one pharmaceutically acceptable carrier. In some embodiments, the pharmaceutical composition is suitable for oral administration. In some embodiments, the composition is provided in the form of a sustained release dosage form.

Pharmaceutically acceptable excipients (carriers) can be liquids, such as water and oils, including those of petroleum, animal, vegetable, or synthetic origin, such as peanut oil, soybean oil, mineral oil, sesame oil and the like. The excipients can be saline, gum acacia, gelatin, starch paste, talc, keratin, colloidal silica, urea and the like. In addition, auxiliary, stabilizing, thickening, lubricating, and coloring agents can be used. In one embodiment the excipients are sterile when administered to an animal. The excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers can be used in preparing solutions, suspensions, emulsions, syrups, and elixirs. The salts and crystalline forms of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained release formulations, suppositories, emulsions, aerosols, sprays, suspensions, or any other form suitable for use. In one embodiment, the composition is in the form of a capsule. Other examples of suitable excipients are described in Remington's Pharmaceutical Sciences 1447 1676 (Alfonso R. Gennaro, ed., 19th ed. 1995).

In one embodiment, the crystalline forms of the invention are formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the salt or crystalline form.

Capsules may contain mixtures of the compounds or pharmaceutically acceptable salts of the compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

When in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the crystalline forms can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the crystalline forms are to be administered by infusion, they can be dispensed, for example, with an infusion bottle containing sterile pharmaceutical grade water or saline. Where the salts and crystalline forms are administered by injection, an ampule of sterile water for injection or saline can be provided so that the ingredients can be mixed prior to administration.

In another embodiment, the crystalline forms can be administered transdermally through the use of a transdermal patch. Transdermal administrations include administrations across the surface of the body and the inner linings of the bodily passages including epithelial and mucosal tissues. Such administrations can be carried out using the present salts and crystalline forms in lotions, creams, foams, patches, suspensions, solutions, and suppositories (e.g., rectal or vaginal).

Transdermal administration can be accomplished through the use of a transdermal patch containing the salt or crystalline form of the invention and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The crystalline forms of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The crystalline forms can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the salt or crystalline form to treat or prevent a 5-HT_{1A}-related disorder in a minimal amount of time. Advantages of controlled- or sustained-release compositions include extended activity of the drug, reduced dosage frequency, and increased compliance by the animal being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound that promptly produces the desired therapeutic or prophylactic effect, and gradually and continually release other amounts of the compound this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the crystalline form delivered is an amount that is effective for treating or preventing a 5-HT_{1A}-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound is administered, the effective dosage amounts correspond to the total amount administered.

The amount of the crystalline form that is effective for treating or preventing a 5-HT_{1A}-related disorder will typically range from about 0.001 mg/kg to about 600 mg/kg of body weight per day, in one embodiment, from about 1 mg/kg to about 600 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.01 mg/kg to about 250 mg/kg, and may be given in a single dose or in two or more divided doses. Variations in the dosage will necessarily occur depending upon the species, weight and condition of the patient being treated and the patient's individual response to the medicament.

In one embodiment, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

In some embodiments, the composition is suitable for oral administration and/or comprises an oral dosage form.

The crystalline forms can be assayed in vitro or in vivo for the desired therapeutic or prophylactic activity prior to use in humans. Animal model systems can be used to demonstrate safety and efficacy.

Pharmaceutical compositions can be prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

### Pharmaceutical Methods

The crystalline forms of the invention are 5-HT_{1A} modulators which are useful in methods of treating various 5-HT_{1A}-related diseases or disorders such as cognition-related disorders or anxiety-related disorders.

The term “5-HT_{1A}-related disorder” as used herein refers to a condition which is mediated through the 5-HT_{1A} receptor. In some embodiments, a 5-HT_{1A}-related disorder is a condition for which it would be beneficial to prevent activation of the 5-HT_{1A} receptor. In other embodiments, a 5-HT_{1A}-related disorder is a condition for which it would be beneficial to activate the 5-HT_{1A} receptor. In one embodiment, a 5-HT_{1A}-related disorder affects the central nervous system (i.e., a CNS-related disorder). Exemplary 5-HT_{1A}-related disorders include, without limitation, depression, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol); behavioral addictions such as an addiction to gambling; and ocular disorders such as glaucoma and ischemic retinopathy; sexual dysfunction associated with drug treatment (e.g., sexual dysfunction associated with SSRI's).

One non-limiting example of a 5-HT_{1A}-related disorder is a cognition-related disorder (e.g., cognitive dysfunction). Exemplary cognition-related disorders include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HUV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the present invention.

Another non-limiting example of a 5-HT_{1A}-related disorder is an anxiety-related disorder. Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

In some embodiments, cognitive-related disorders can include improving cognitive function or inhibiting cognitive deficits. Examples of improvements in cognitive function include, without limitation, memory improvement and retention of learned information. Accordingly, the compounds are useful for slowing the loss of memory and cognition and for maintaining independent function for patients afflicted with a cognition-related disorder. Accordingly, the salts and crystalline forms of the present invention are useful for improving cognitive function. Further examples of cognition-related disorders include dementia, Parkinson's disease, Huntington's disease, Alzheimer's disease, cognitive deficits associated with Alzheimer's disease, mild cognitive impairment, and schizophrenia.

In some embodiments, anxiety-related disorders include attention deficit disorder, obsessive compulsive disorder, substance addiction, withdrawal from substance addiction, premenstrual dysphoric disorder, social anxiety disorder, anorexia nervosa, and bulimia nervosa.

The crystalline forms of the invention are further useful for treating Alzheimer's disease. In some embodiments, the method for treating Alzheimer's disease includes administering a second therapeutic agent. In some embodiments, the second therapeutic agent is an anti-depressant agent, an anti-anxiety agent, an anti-psychotic agent, or a cognitive enhancer.

The crystalline forms of the invention are further useful for treating mild cognitive impairment (MCI). In some embodiments, the method for treating MCI includes administering a second therapeutic agent. In some embodiments, the second therapeutic agent is an anti-depressant agent, an anti-anxiety agent, an anti-psychotic agent, or a cognitive enhancer.

The crystalline forms of the invention are further useful for treating depression. In some embodiments, the method for treating depression includes administering a second therapeutic agent. In some embodiments, the second therapeutic agent is an anti-depressant agent, an anti-anxiety agent, an anti-psychotic agent, or a cognitive enhancer.

The crystalline forms of the invention are further useful for treating sexual dysfunction, such as sexual dysfunction associated with drug treatment (e.g., with an antidepressant, an antipsychotic, or an anticonvulsant).

In certain embodiments, the drug treatment associated with sexual dysfunction involves a selective serotonin reuptake inhibitor (SSRI) (for example, fluoxetine, citalopram, escitalopram oxalate, fluvoxamine maleate, paroxetine, or sertraline), a tricyclic antidepressant (for example, desipramine, amitriptyline, amoxipine, clomipramine, doxepin, imipramine, nortriptyline, protriptyline, trimipramine, dothiepin, butriptyline, iprindole, or lofepramine), an aminoketone class compound (for example, bupropion). In some embodiments, the drug is a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT1A agonist (for example, buspirone), a 5-HT2A receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenyloin, primidone, or carbamazepine. In some cases, the patient in need of treatment for sexual dysfunction is being treated with at least two drugs that are antidepressant drugs, antipsychotic drugs, anticonvulsant drugs, or a combination thereof.

In some embodiments of the invention, the sexual dysfunction comprises a deficiency in penile erection.

In some embodiments, the crystalline forms are effective for ameliorating sexual dysfunction in an animal model of sexual dysfunction associated with drug treatment, for example, in an animal model of sexual dysfunction that is an antidepressant drug-induced model of sexual dysfunction.

The crystalline forms of the invention are further useful for improving sexual function in a patient.

As used herein, the term “patient” refers to any animal, including mammals, preferably mice, rats, other rodents, rabbits, dogs, cats, swine, cattle, sheep, horses, or primates, and most preferably humans. In some embodiments, the patient is in need of treatment.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

### Administration, Compositions, and Dosage Forms

The crystalline forms of the invention can be administered neat or as a component of a composition that comprises a physiologically acceptable carrier or vehicle. A pharmaceutical composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

The present pharmaceutical compositions can be administered orally. The crystalline forms of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound or a pharmaceutically acceptable salt of the compound into the bloodstream. The mode of administration is left to the discretion of the practitioner.

In one embodiment, the crystalline forms of the invention are administered orally.

In another embodiment, the crystalline forms of the invention are administered intravenously.

In another embodiment, it may be desirable to administer the crystalline forms of the invention locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the crystalline forms of the invention into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the crystalline forms can be formulated as a suppository, with traditional binders and excipients such as triglycerides.

In another embodiment, the crystalline forms of the invention can be delivered in a vesicle, in particular a liposome (see Langer, Science 249:1527-1533 (1990) and Treat et al., Liposomes in the Therapy of Infectious Disease and Cancer 317-327 and 353-365 (1989)).

In yet another embodiment, the crystalline forms of the invention can be delivered in a controlled-release system or sustained-release system (see, e.g., Goodson, in Medical Applications of Controlled Release, vol. 2, pp. 115 138 (1984)). Other controlled or sustained-release systems discussed in the review by Langer, Science 249:1527 1533. (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

### Combination Therapy

The crystalline forms of the invention can be administered to a patient in combination with a therapeutically effective amount of one or more further therapeutic agents. Effective amounts of further therapeutic agents are well known to those skilled in the art. It is well within the skilled artisan's purview to determine the other therapeutic agent's optimal effective amount range. The crystalline form and the other therapeutic agent can act additively or, in one embodiment, synergistically. In one embodiment of the invention, where another therapeutic agent is administered to an animal, the effective amount of crystalline form is less than its effective amount would be where the other therapeutic agent is not administered. In this case, without being bound by theory, it is believed that the crystalline form and the other therapeutic agent can act synergistically. In some cases, the patient in need of treatment is being treated with one or more other therapeutic agents. In some cases, the patient in need of treatment is being treated with at least two other therapeutic agents.

In one embodiment, the other therapeutic agent is selected from one or more of the following: anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the active compounds of this invention include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that can be used in combination with the active compounds of this invention include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenyloin, primidone, or carbamazepine.

Cognitive enhancers that can be used in combination with the active compounds of this invention include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5HT receptor modulators, 5HT_{1A} antagonists or agonists (including without limitation lecozotan and compounds described in U.S. Pat. Nos. 6,465,482, 6,127,357, 6,469,007, and 6,586,436, and in PCT Publication No. WO 97/03982) and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) IV inhibitors (e.g., roflumilast, arofylline, cilomilast, rolipram, RO-20-1724, theophylline, denbufylline, ARIFLO, ROFLUMILAST, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In one embodiment, the crystalline form of the invention and cognitive enhancer act additively or, in one embodiment, synergistically. In one embodiment, where a cognitive enhancer and a crystalline form of the invention are co-administered to an animal, the effective amount of or crystalline form of the invention is less than its effective amount would be where the cognitive enhancer agent is not administered. In one embodiment, where a cognitive enhancer and a crystalline form of the invention are co-administered to an animal, the effective amount of the cognitive enhancer is less than its effective amount would be where the crystalline form of the invention is not administered. In one embodiment, a cognitive enhancer and a crystalline form of the invention are co-administered to an animal in doses that are less than their effective amounts would be where they were no co-administered. In these cases, without being bound by theory, it is believed that the compound or a pharmaceutically acceptable salt of the compound and the cognitive enhancer act synergistically.

In one embodiment, the other therapeutic agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In one embodiment, the crystalline form is administered concurrently with at least one further therapeutic agent.

In one embodiment, a composition comprising an effective amount of the crystalline form and an effective amount of at least one further therapeutic agent within the same composition can be administered.

In another embodiment, a composition comprising an effective amount of the crystalline form and a separate composition comprising an effective amount of a further therapeutic agent can be concurrently administered. In another embodiment, an effective amount of the crystalline form is administered prior to or subsequent to administration of an effective amount of a further therapeutic agent. In this embodiment, the crystalline form is administered while the other therapeutic agent exerts its therapeutic effect, or the other therapeutic agent is administered while the crystalline form exerts its preventative or therapeutic effect for treating or preventing a 5-HT_{1A}-related disorder.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of 6-methoxy-8-[4-(1-(5-fluoro)-quinolin-8-yl-piperidin-4-yl)-piperazin-1-yl]-quinoline (Form A)

### Step 1

### 5-Fluoro-8 chloroquinoline

To a mixture of (5.0 g) 2-chloro-5-fluoroaniline (commercially available, 6.0 g), glycerol (6.0 g) and m-nitrobenzene sulfonic acid sodium salt (11.0 g), was added 20 mL of 70% sulfuric acid dropwise. The reaction temperature was raised to 140° C. for 2 h. The mixture was then cooled, poured on ice water and filtered through celite. The filtrate was neutralized with NaOH and extracted with CH₂Cl₂. The combined organic layers were dried over anhydrous MgSO₄ and concentrated on a rotary evaporator. The crude product was purified by flash chromatography on silica gel using 100% CH₂Cl₂ to give 3.7 g of the desired product of a yellow solid; MP=74-76° C.; MS (ES) m/z (relative intensity): 182 (M+H)⁺ (100).

### Step 2

### 8-(1,4-Dioxa-8-azaspiro[4,5]dec-5-yl)-5-fluoroquinoline

To a solution of 5-fluoro-8-chloroquinoline (Step 1, 1.12 g) in 20 mL of anhydrous tetrahydrofuran, was added 0.085 g of tris(dibenzylideneacetone)dipalladium(0) (Pd₂(dba)₃, 0.085 g), sodium tert-butoxide (0.83 g), 2-dicyclohexylphosphino-2′-(N,N-dimethylamino)-biphenyl (CYMAP, 0.036 g), and 1,4-dioxo-8-azaspiro-4,5-decane (1.05 g). The mixture was refluxed for 6 hours under a nitrogen atmosphere. The reaction mixture was then cooled to room temperature, diluted with ether, filtered through celite and concentrated on a rotary evaporator. The crude material was then purified by flash chromatography on silica gel using hexane/ethyl acetate to give 0.700 g of the desired product as a brown oil; MS (ES) m/z (relative intensity): 289 (M+H)⁺ (100).

### Step 3

### 1-(5-Fluoroquinolin-8-yl)piperidin-4-one

A solution of 8-(1,4-dioxa-8-azaspiro[4,5]dec-5-yl)-5-fluoroquinoline (Step 2, 2.1 g)) in 10 mL of 1:1 tetrahydrofuran/2N aqueous HCl was stirred at room temperature overnight. The reaction mixture was diluted with water, made basic with 1N aqueous NaOH and extracted with CH₂Cl₂. The combined organic layers were dried over anhydrous MgSO₄, filtered and concentrated on a rotary evaporator to give 1.68 g of the desired product as a yellow solid which was pure enough to use in subsequent reactions; MS m/z=245 [M+H]⁺.

### Step 4

### 8-Chloro-6-hydroxyquinoline

In a 500 mL 3-necked flask equipped with a mechanical stirrer, a reflux condenser, were added in order ferrous sulfate (2.0 g), 4-amino-3-chlorophenol hydrochloride (6.4 g, commercially available), nitrobenzene (2.9 mL) and a solution of boric acid (3.0 g) in glycerol (16 g). Then concentrated sulfuric acid (9 mL) was added drop by drop with cooling. The ice bath was removed and replaced by an oil bath and the mixture was heated cautiously to 120° C. for 2 hours, then at 150° C. and kept stirring under this temperature for 20 hours. After cooling, the reaction was poured on crushed ice and the resulting solution was neutralized with K₂CO₃. The product separated as a light brown solid that was filtered off, washed with water and hexanes and dried in a vacuum oven (35° C.) overnight giving 7 g (77%) of the desired product. MS (ES) m/z (relative intensity): 180 (M++−H, 100).

### Step 5

### 8-Chloro-6-methoxyquinoline

To a solution of 3.3 g of 8-chloro-6-hydroxyquinoline (Step 4, 3.3 g) in dimethylformamide was added K₂CO₃ (3.8 g), followed by iodomethane (5.2 g). The mixture was stirred at room temperature overnight. Water was then added and the aqueous mixture was extracted with CH₂Cl₂. The combined organic layers were dried over anhydrous MgSO₄, filtered and concentrated on a rotary evaporator. The crude product was purified by flash chromatography on silica gel using 100% CH₂Cl₂, to give 2.2 g of the desired product as a beige solid; MP=74-75° C.; MS (ES) m/z (relative intensity): 194 (M+H)⁺ (100).

### Step 6

### 6-Methoxy-8-[1-(tert-butoxycarbonyl)-4-piperazino]quinoline

To a mixture of 8-chloro-6-methoxyquinoline (Step 5, 2.7 g) in anhydrous tetrahydrofuran, was added tris(dibenzylideneacetone)dipalladium(0) (Pd₂(dba)₃, 0.064 g) sodium tert-butoxide (1.9 g), 2-dicyclohexylphosphino-2′-(N,N-dimethylamino)biphenyl (CYMAP, 0.08 g) and tert-butoxycarbonylpiperazine (3.4 g). The mixture was refluxed for 5 hours under a nitrogen atmosphere. The reaction was then cooled to room temperature, diluted with ether, filtered through Celite and concentrated on a rotary evaporator. The crude material was purified by flash chromatography using 100% CH₂Cl₂ to give 4.0 g of the desired product as a beige solid; mp=92-93° C.; MS (ES) m/z (relative intensity): 344 (M⁺+H) (100).

### Step 7

### 6-Methoxy-8-piperazinoquinoline

To a solution of 6-methoxy-8-[1-(tert-butoxycarbonyl)-4-piperazino]quinoline (Step 6, 4.0 g) in 20 mL of dDioxane was added 10 mL of 4 N HCl/dioxane. The mixture was stirred at room temperature overnight. The resulting precipitate was collected by vacuum filtration, dissolved in water, neutralized with aqueous sodium hydroxide and extracted with CH₂Cl₂. The combined organic layers were dried over anhydrous Na₂SO₄, filtered and concentrated on a rotary evaporator to give 2.8 g of the desired product as a beige solid; MP=105-107° C.; MS (ES) m/z (relative intensity): 244 (M+H)⁺ (100).

### Step 8

### 6-Methoxy-8-[4-(1-(5-fluoro)-quinolin-8-yl-piperidin-4-yl)-piperazin-1-yl]-quinoline

1-(5-Fluoro-quinolin-8-yl)-piperidin-4-one (Step 3, 4.1 g) and 6-methoxy-8-piperazinoquinoline (Step 7, 4.3 g) were stirred in 20 mL of anhydrous methanol. Then 1.1 eq (1.2 gm) of sodium cyanoborohydride was added and the reaction was stirred overnight at room temperature. The solvent was stripped off and the residue was taken up in ethyl acetate and washed with water. The organic layer was dried over anhydrous sodium sulfate, filtered and stripped to a yellow solid giving 7.4 g. The crude product was purified by preparative HPLC (4% ethanol in hexane). The resulting solution was filtered and the solvent stripped (by rotary evaporation) from the filtrate to yield 1.27 g of yellow solid which was characterized as Form A by XRPD. MS [M+H]+ 472.2. m.p. 198° C. Purity 94-98%.

### Example 2

### Preparation of 6-methoxy-8-[4-(1-(5-fluoro)-quinolin-8-yl-piperidin-4-yl)-piperazin-1-yl]-quinoline (Form B)

6-Methoxy-8-[4-(1-(5-fluoro)-quinolin-8-yl-piperidin-4-yl)-piperazin-1-yl]-quinoline Form A was dissolved in DMSO. An off-white powder precipitated upon addition of water antisolvent. The solid was collected by vacuum filtration and air-dried. The dried solid was characterized by XRPD as Form B.

### Example 3

### Preparation of 6-methoxy-8-[4-(1-(5-fluoro)-quinolin-8-yl-piperidin-4-yl)-piperazin-1-yl]-quinoline (Form C)

6-Methoxy-8-[4-(1-(5-fluoro)-quinolin-8-yl-piperidin-4-yl)-piperazin-1-yl]-quinoline Form A was dissolved in a 1:1 mixture of methyl ethyl ketone and dioxane. Upon evaporation of solvent in an open vial under ambient conditions, plate-like crystals were obtained that were characterized as Form C by XRPD.

### Example 4

### Preparation of 6-methoxy-8-[4-(1-(5-fluoro)-quinolin-8-yl-piperidin-4-yl)-piperazin-1-yl]-quinoline (Form D)

6-Methoxy-8-[4-(1-(5-fluoro)-quinolin-8-yl-piperidin-4-yl)-piperazin-1-yl]-quinoline Form A was dissolved in ethyl acetate. Upon evaporation of the solvent in an open vial under ambient conditions, yellow prismatic crystals were obtained that were characterized as Form D by XRPD.

### Example 5

### Preparation of 6-methoxy-8-[4-(1-(5-fluoro)-quinolin-8-yl-piperidin-4-yl)-piperazin-1-yl]-quinoline (Form E)

6-Methoxy-8-[4-(1-(5-fluoro)-quinolin-8-yl-piperidin-4-yl)-piperazin-1-yl]-quinoline Form A was slurried in tetrahydrofuran at 50° C. for about 48 hours. The resulting solid was characterized as Form E by XRPD.

### Example 6

### Powder X-Ray Diffraction

Powder X-ray diffraction patterns were obtained on a Rigaku Miniflex Diffraction System (Rigaku MSC inc.). The powder samples were deposited on a zero-background polished silicon sample holder. A normal focus copper X-ray tube at 0.45 kW equipped with a Ni Kβ filter scanning at 1 degrees/minute from 3.00 to 40.00 degree 2-theta was used as the x-ray source. In some instance, a faster scanning rate of 2 degrees/minute was applied. The data processing was done using Jade 6.0 software.

### Example 7

### Differential Scanning Calorimetry

DSC data was collected using a Q1000 DSC (TA instruments). Typically 3-5 mg of sample was used in a hermetically sealed aluminum pan (no pin-hole). The sample was heated from 40° C.-300° C. at a ramp rate of 10° C./min. The heat flow data was analyzed using Universal Analysis software (TA instruments).

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A crystalline form (Form A) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 9.6 and about 13.1, and having a DSC thermogram which is characterized by an endothermic peak at about 196° C.

2. The crystalline form of claim 1 further comprising a characteristic peak, in terms of 2θ (°), at about 22.6.

3. The crystalline form of claim 2 further comprising a characteristic peak, in terms of 2θ (°), at about 24.0.

4. A crystalline form (Form A) of 6-methoxy-8-[4-(1-(5-fluoro)-quinolin-8-yl-piperidin-4-yl)-piperazin-1-yl]-quinoline having an X-ray powder diffraction pattern comprising at least three characteristic peaks, in terms of 2θ (°), selected from about 9.6, about 13.1, about 22.6, about 24.0, about 19.5, and about 20.3, and having a DSC thermogram which is characterized by an endothermic peak at about 196° C.

5. A crystalline form (Form A) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 1.

6. The crystalline form (Form A) of 6-methoxy-8-[4-(1-(5-fluoro)-quinolin-8-yl-piperidin-4-yl)-piperazin-1-yl]-quinoline having a DSC thermogram which is characterized by an endothermic peak at about 196° C.

7. The crystalline form of claim 6 having a DSC thermogram substantially as shown in FIG. 6.

8. A crystalline form (Form B) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 9.0 and about 15.9.

9. The crystalline form of claim 8 further comprising a characteristic peak, in terms of 2θ (°), at about 22.4.

10. The crystalline form of claim 9 further comprising a characteristic peak, in terms of 2θ (°), at about 25.2.

11. A crystalline form (Form B) of 6-methoxy-8-[4-(1-(5-fluoro)-quinolin-8-yl-piperidin-4-yl)-piperazin-1-yl]-quinoline having an X-ray powder diffraction pattern comprising a characteristic peak, in terms of 2θ (°), of about 9.0 and at least three characteristic peaks, in terms of 20, selected from about 12.6, about 13.4, about 15.9, about 22.4, and about 25.2.

12. A crystalline form (Form B) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 2.

13. The crystalline form of claim 12 having a DSC thermogram substantially as shown in FIG. 6.

14. A crystalline form (Form C) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 9.6 and about 13.1, and having a DSC thermogram which is characterized by an endothermic peak at about 192° C.

15. The crystalline form of claim 14 further comprising a characteristic peak, in terms of 2θ (°), at about 22.4.

16. The crystalline form of claim 15 further comprising a characteristic peak, in terms of 2θ (°), at about 23.8.

17. A crystalline form (Form C) of 6-methoxy-8-[4-(1-(5-fluoro)-quinolin-8-yl-piperidin-4-yl)-piperazin-1-yl]-quinoline having an X-ray powder diffraction pattern comprising at least three characteristic peaks, in terms of 2θ (°), selected from about 9.6, about 13.1, about 15.7, about 19.4, about 22.4, about 22.8, about 23.8, and having a DSC thermogram which is characterized by an endothermic peak at about 192° C.

18. A crystalline form (Form C) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 3.

19. The crystalline form (Form C) of 6-methoxy-8-[4-(1-(5-fluoro)-quinolin-8-yl-piperidin-4-yl)-piperazin-1-yl]-quinoline having a DSC thermogram which is characterized by an endothermic peak at about 192° C.

20. The crystalline form of claim 19 having a DSC thermogram substantially as shown in FIG. 6.

21. A crystalline form (Form D) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 12.2 and about 12.8.

22. The crystalline form of claim 21 further comprising a characteristic peak, in terms of 2θ (°), at about 13.0.

23. The crystalline form of claim 22 further comprising a characteristic peak, in terms of 2θ (°), at about 19.6.

24. A crystalline form (Form D) of 6-methoxy-8-[4-(1-(5-fluoro)-quinolin-8-yl-piperidin-4-yl)-piperazin-1-yl]-quinoline having an X-ray powder diffraction pattern comprising a characteristic peak, in terms of 2θ (°), of about 12.2 and at least three characteristic peaks, in terms of 20, selected from about 12.8, about 13.0, about 18.8, about 19.6, about 22.4.

25. A crystalline form (Form D) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 4.

26. A crystalline form (Form D) of 6-methoxy-8-[4-(1-(5-fluoro)-quinolin-8-yl-piperidin-4-yl)-piperazin-1-yl]-quinoline having a DSC thermogram substantially as shown in FIG. 6.

27. A crystalline form (Form E) of 6-methoxy-8-[4-(1-(5-fluoro)-quinolin-8-yl-piperidin-4-yl)-piperazin-1-yl]-quinoline having an X-ray powder diffraction pattern comprising characteristic peaks, in terms of 2θ (°), at about 12.3 and about 19.7.

28. The crystalline form of claim 27 further comprising a characteristic peak, in terms of 2θ (°), at about 18.9.

29. The crystalline form of claim 28 further comprising a characteristic peak, in terms of 2θ (°), at about 21.8.

30. A crystalline form (Form E) of 6-methoxy-8-[4-(1-(5-fluoro)-quinolin-8-yl-piperidin-4-yl)-piperazin-1-yl]-quinoline having an X-ray powder diffraction pattern comprising a characteristic peak, in terms of 2θ (°), of about 12.3 and at least three characteristic peaks, in terms of 20, selected from about 12.9, about 18.9, about 19.7, about 21.8, about 22.1.

31. A crystalline form (Form E) of 6-methoxy-8-[4-(1-(5-fluoro)-quinolin-8-yl-piperidin-4-yl)-piperazin-1-yl]-quinoline having an X-ray powder diffraction pattern substantially as shown in FIG. 5.

32. A crystalline form (Form E) of 6-methoxy-8-[4-(1-(5-fluoro)-quinolin-8-yl-piperidin-4-yl)-piperazin-1-yl]-quinoline having a DSC thermogram substantially as shown in FIG. 6.

33. A method for treating a 5-HT_{1A}-related disorder in a patient in need thereof, the method comprising administering to said patient a therapeutically effective amount a crystalline form of claim 1, 8, 14, 21, or 27.

34. The method of claim 33 wherein the 5-HT_{1A}-related disorder is a cognition-related disorder or an anxiety-related disorder.

35. The method of claim 34 wherein the cognition-related disorder is dementia, Parkinson's disease, Huntington's disease, Alzheimer's disease, cognitive deficits associated with Alzheimer's disease, mild cognitive impairment, or schizophrenia.

36. The method of claim 34, wherein the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

37. The method of claim 34 further comprising administering a second therapeutic agent.

38. The method of claim 37 wherein the second therapeutic agent is an anti-depressant agent, an anti-anxiety agent, anti-psychotic agent, or a cognitive enhancer.

39. The method of claim 37 wherein the second therapeutic agent is a selective serotonin reuptake inhibitor.

40. The method of claim 37 wherein the second therapeutic agent is fluoxetine, fluvoxamine, paroxetine, sertaline, clonazepam, diazepam, buspirone, haloperidol, olanzapine, or clozapine.

41. The method of claim 37 wherein the second therapeutic agent is a cholinesterase inhibitor.

42. The method of claim 37 wherein the second therapeutic agent is tacrine, donepezil, rivastigmine, or galantamine.

43. A method for treating Alzheimer's disease in a patient in need thereof, the method comprising administering to said patient a therapeutically effective amount of a crystalline form of claim 1, 8, 14, 21, or 27.

44. The method of claim 43 further comprising administering a second therapeutic agent.

45. The method of claim 44 wherein the second therapeutic agent is an anti-depressant agent, an anti-anxiety agent, anti-psychotic agent, or a cognitive enhancer.

46. The method of claim 44 wherein the second therapeutic agent is a selective serotonin reuptake inhibitor.

47. The method of claim 44 wherein the second therapeutic agent is fluoxetine, fluvoxamine, paroxetine, sertaline, clonazepam, diazepam, buspirone, haloperidol, olanzapine, or clozapine.

48. The method of claim 44 wherein the second therapeutic agent is a cholinesterase inhibitor.

49. The method of claim 44 wherein the second therapeutic agent is tacrine, donepezil, rivastigmine, or galantamine.

50. A method for treating mild cognitive impairment (MCI) in a patient in need thereof, the method comprising administering to said patient a therapeutically effective amount of a crystalline form of claim 1, 8, 14, 21, or 27.

51. The method of claim 50 further comprising administering a second therapeutic agent.

52. The method of claim 51 wherein the second therapeutic agent is an anti-depressant agent, an anti-anxiety agent, anti-psychotic agent, or a cognitive enhancer.

53. The method of claim 51 wherein the second therapeutic agent is a selective serotonin reuptake inhibitor.

54. The method of claim 51 wherein the second therapeutic agent is fluoxetine, fluvoxamine, paroxetine, sertaline, clonazepam, diazepam, buspirone, haloperidol, olanzapine, or clozapine.

55. The method of claim 51 wherein the second therapeutic agent is a cholinesterase inhibitor.

56. The method of claim 51 wherein the second therapeutic agent is tacrine, donepezil, rivastigmine, or galantamine.

57. A method for treating depression in a patient in need thereof, the method comprising administering to said patient a therapeutically effective amount of a crystalline form of claim 1, 8, 14, 21, or 27.

58. The method of claim 57 further comprising administering a second therapeutic agent.

59. The method of claim 58 wherein the second therapeutic agent is an anti-depressant agent, an anti-anxiety agent, anti-psychotic agent, or a cognitive enhancer.

60. The method of claim 58 wherein the second therapeutic agent is a selective serotonin reuptake inhibitor.

61. The method of claim 58 wherein the second therapeutic agent is fluoxetine, fluvoxamine, paroxetine, sertaline, clonazepam, diazepam, buspirone, haloperidol, olanzapine, or clozapine.

62. The method of claim 58 wherein the second therapeutic agent is a cholinesterase inhibitor.

63. The method of claim 58 wherein the second therapeutic agent is tacrine, donepezil, rivastigmine, or galantamine.

64. A method for treating sexual dysfunction associated with drug treatment in a patient in need thereof, the method comprising administering to the patient a therapeutically effective amount of a crystalline form of claim 1, 8, 14, 21, or 27.

65. The method of claim 64 wherein the drug treatment is antidepressant drug treatment, antipsychotic drug treatment, or anticonvulsant drug treatment.

66. A method of improving sexual function in a patient in need thereof, the method comprising administering to the patient an effective amount of crystalline form of claim 1, 8, 14, 21, or 27.

67. A composition comprising a crystalline form of claim 1, 8, 14, 21, or 27 and at least one pharmaceutically acceptable carrier.

68. The composition of claim 67 further comprising a second therapeutic agent.

69. The composition of claim 68 wherein said second therapeutic agent is an anti-depressant agent, an anti-anxiety agent, anti-psychotic agent, or a cognitive enhancer.

70. The composition of claim 68 wherein said second therapeutic agent is a selective serotonin reuptake inhibitor.

71. The composition of claim 68 wherein said second therapeutic agent is fluoxetine, fluvoxamine, paroxetine, sertaline, clonazepam, diazepam, buspirone, haloperidol, olanzapine, or clozapine.

72. The composition of claim 68 wherein said second therapeutic agent is a cholinesterase inhibitor.

73. The composition of claim 68 wherein said second therapeutic agent is tacrine, donepezil, rivastigmine, or galantamine.
